(19) United States
(12) Patent Application Publication
(10) Pub. No.: US 2011/0189169 A1
(43) Pub. Date: Aug. 4, 2011

(54) COMBINATION OF HGF INHIBITOR AND PTEN AGONIST TO TREAT CANCER

(75) Inventors: Roger Abounader, Charlottesville, VA (US); Yunqing Li, Charlottesville, VA (US)

(73) Assignees: GALAXY BIOTECH, LLC, Cupertino, CA (US); UNIVERSITY OF VIRGINIA PATENT FOUNDATION, Charlotteville, VA (US)

(21) Appl. No.: 12/935,581

(22) PCT Filed: Apr. 9, 2009
(86) PCT No.: PCT/US09/40122

§ 371 Date: Apr. 15, 2011

(30) Foreign Application Priority Data
Apr. 11, 2008 (US) 61044446

(51) Int. Cl. A61K 39/395; A61P 35/00

(52) U.S. Cl. 424/133.1; 424/145.1

(57) ABSTRACT

The present invention is directed toward a method of treating cancer by administering to a patient an inhibitor of Hepatocyte Growth Factor and an agonist of PTEN.

### CROSS-REFERENCE TO RELATED APPLICATIONS

This application claims benefit under 35 U.S.C. §119(e) of U.S. Patent Application No. 61/044,446 filed Apr. 11, 2008, which is herewith incorporated in its entirety for all purposes.

### STATEMENT OF GOVERNMENT INTEREST

The invention described in this application was made in part with funding by Grant RO1 NS045209 from the National Institutes of Health. The US Government has certain rights in this invention.

### FIELD OF THE INVENTION

The present invention relates generally to the treatment of cancer, and more particularly, for example, to treatment of cancer with an agent that inhibits Hepatocyte Growth Factor together with a PTEN agonist.

### BACKGROUND OF THE INVENTION

Human Hepatocyte Growth Factor (HGF) is a multifunctional heterodimeric polypeptide produced by mesenchymal cells. HGF has been shown to stimulate angiogenesis, morphogenesis and motogenesis, as well as the growth and scattering of various cell types (Bussolino et al., J. Cell. Biol. 119: 629, 1992; Zarnegar and Michalopoulos, J. Cell. Biol. 129:1177, 1995; Matsumoto et al., Ciba. Found. Symp. 212:198, 1997; Birchmeier and Gherardi, Trends Cell. Biol. 8:404, 1998; Xin et al. Am. J. Pathol. 158:1111, 2001). The pleiotropic activities of HGF are mediated through its receptor, a transmembrane tyrosine kinase encoded by the proto-oncogene cMet. In addition to regulating a variety of normal cellular functions, HGF and its receptor c-Met have been shown to be involved in the initiation, invasion and metastasis of tumors (Jeffers et al., J. Mol. Med. 74:505, 1996; Comoglio and Trusolino, J. Clin. Invest. 109:857, 2002). HGF/cMet are coexpressed, often over-expressed, on various human solid tumors including tumors derived from lung, colon, rectum, stomach, kidney, ovary, skin, multiple myeloma and thyroid tissue (Prat et al., Int. J. Cancer 49:323, 1991; Chan et al., Oncogene 2:593, 1988; Weidner et al., Am. J. Respir. Cell. Mol. Biol. 8:229, 1993; Derksen et al., Blood 99:1405, 2002). HGF acts as an autocrine (Rong et al., Proc. Natl. Acad. Sci. USA 91:4731, 1994; Koochekpour et al., Cancer Res. 57:5391, 1997) and paracrine growth factor (Weidner et al., Am. J. Respir. Cell. Mol. Biol. 8:229, 1993) and anti-apoptotic regulator (Gao et al., J. Biol. Chem. 276:47257, 2001) for these tumors.

HGF is a 102 kDa protein with sequence and structural similarity to plasminogen and other enzymes of blood coagulation (Nakamura et al., Nature 342:440, 1989; Weidner et al., Am. J. Respir. Cell. Mol. Biol. 8:229, 1993, each of which is incorporated herein by reference). Human HGF is synthesized as a 728 amino acid precursor (preproHGF), which undergoes intracellular cleavage to an inactive, single chain form (proHGF) (Nakamura et al., Nature 342:440, 1989; Rosen et al., J. Cell. Biol. 127:1783, 1994). Upon extracellular secretion, proHGF is cleaved to yield the biologically active disulfide-linked heterodimeric molecule composed of an α-subunit and β-subunit (Nakamura et al., Nature 342:440, 1989; Naldini et al., EMBO J. 11:4825, 1992). The α-subunit contains 440 residues (69 kDa with glycosylation), consisting of the N-terminal hairpin domain and four kringle domains. The β-subunit contains 234 residues (34 kDa) and has a serine protease-like domain, which lacks proteolytic activity. Cleavage of HGF is required for receptor activation, but not for receptor binding (Hartmann et al., Proc. Natl. Acad. Sci. USA 89:11574, 1992; Lokker et al., J. Biol. Chem. 268:17145, 1992). HGF contains 4 putative N-glycosylation sites, 1 in the α-subunit and 3 in the β-subunit. HGF has 2 unique cell specific binding sites: a high affinity (Kd=2×10⁻¹⁰ M) binding site for the cMet receptor and a low affinity (Kd=10⁻⁹ M) binding site for heparin sulfate proteoglycans (HSPG), which are present on the cell surface and extracellular matrix (Naldini et al., Oncogene 6:501, 1991; Bardelli et al., J. Biotechnol. 37:109, 1994; Sakata et al., J. Biol. Chem., 272:9457, 1997).

cMet is a member of the class IV protein tyrosine kinase receptor family. The full length cMet gene was cloned and identified as the cMet proto-oncogene (Cooper et al., Nature 311:29, 1984; Park et al., Proc. Natl. Acad. Sci. USA 84:6379, 1987). The cMet receptor is initially synthesized as a single chain, partially glycosylated precursor, p170^{(MET)} (Park et al., Proc. Natl. Acad. Sci. USA 84:6379, 1987; Giordano et al., Nature 339:155, 1989; Giordano et al., Oncogene 4:1383, 1989; Bardelli et al., J. Biotechnol. 37:109, 1994). Upon further glycosylation, the protein is proteolytically cleaved into a heterodimeric 190 kDa mature protein (1385 amino acids), consisting of the 50 kDa α-subunit (residues 1-307) and the 145 kDa β-subunit. The cytoplasmic tyrosine kinase domain of the β-subunit is involved in signal transduction.

Several different approaches have been investigated to obtain HGF inhibitors, i.e. antagonists. Such inhibitors include truncated HGF proteins such as NK1 (N terminal domain plus kringle domain 1; Lokker et al., J. Biol. Chem. 268:17145, 1993); NK2 (N terminal domain plus kringle domains 1 and 2; Chan et al., Science 254:1382, 1991); and NK4 (N-terminal domain plus four kringle domains), which was shown to partially inhibit the primary growth and metastasis of murine lung tumor LLC in a nude mouse model (Kuba et al., Cancer Res. 60:6737, 2000)

As another approach, Dodge (Master's Thesis, San Francisco State University, 1998) generated antagonist anti-cMet monoclonal antibodies (mAbs). One mAb, 5D5, exhibited strong antagonistic activity in ELISA, but induced a proliferative response of cMet-expressing BAF-3 cells, presumably due to dimerization of the membrane receptors. For this reason, a single-domain form of the anti-cMet mAb 5D5 has been developed as an antagonist (Nguyen et al., Cancer Gene Ther. 10:840, 2003).

Cao et al., Proc. Natl. Acad. Sci. USA 98:7443, 2001, reported that the administration of a cocktail of three anti-HGF mAbs, which were selected based upon their ability to inhibit the scattering activity of HGF in vitro, were able to inhibit the growth of human tumors in the xenograft nude mouse model.

More recently, several neutralizing (inhibitory) anti-HGF mAbs have been reported including L2G7 (Kim et al., Clin Cancer Res 12:1292, 2006 and U.S. Pat. No. 7,220,410), HuL2G7 (WO 07115049 A2), the human mAbs described in WO 2005/017107 A2, and the HGF binding proteins described in WO 07143090 A2 or WO 07143098 A2. It has also been reported that the anti-HGF mAb L2G7, when administered systemically, can strongly inhibit growth or even induce regression of orthotopic (intracranial) glioma xenografts and prolong animal survival (Kim et al., op. cit. and WO 06130773 A2).

PTEN (Phosphatase and tensin homologue deleted on chromosome 10) is a widely expressed tumor suppressor (Li et al., J. Cell. Biochem. 102:1368, 2007). PTEN is a phospholipid phosphatase specific for the 3-position of the inositol ring in the signaling molecule phosphatidylinositol triphosphate, PI(3, 4, 5)P3, that is it converts PI(3, 4, 5)P3 to PI(4, 5)P2 (or written more simply, PIP3 to PIP2). This is the opposite reaction to that catalyzed by phosphatidylinositol 3-kinases (PI3Ks). The PI3Ks are a large family of proteins activated by many cellular receptors (e.g., growth factor receptors upon ligand binding) that in turn activate the Akt protein via PIP3. Akt then directly or indirectly activates many other proteins, notably mTOR (mammalian target of rapamycin), leading for example to protein synthesis, cell proliferation and cell survival. Thus, the PI3K/Akt pathway plays a key, generally positive, role in regulating cell cycle progression, cell proliferation and cell survival (Jiang et al., Biochim. Biophys. Acta 1784:150, 2008). PTEN can control and down-regulate this pathway by reducing levels of the intermediate signaling molecule PIP3. PTEN also possesses phosphatase-independent tumor suppressive functions.

Because of the key role of the PI3K/Akt pathway in cell proliferation and cell survival, mutations in components of this pathway or in PTEN are found in many tumors. In particular, deletions or mutations of the PTEN gene, or commonly mutation of one allele and deletion of the other, are found at high frequency in many types of cancer including melanoma, endometrial, breast, prostate, ovarian, lung, bladder, gastric, cervix, head and neck, renal and brain tumors (Li et al., op. cit.; Jiang et al., op. cit.). PTEN is thus an important tumor suppressor, since the inactivated PTEN is unable to regulate the PI3K/Akt pathway. In particular, loss or mutation of PTEN is very common in glioblastoma, a type of brain tumor, with somatic mutations of PTEN detected in over 40% of these tumors and PTEN protein expression very low or absent in two-thirds of the tumors (Teng et al., Cancer Res. 57:5221, 1997; Wang et al., Cancer Res. 57:4183, 1997; Sano et al., Cancer Res. 59:1820, 1999).

In an attempt to treat cancer, a number of inhibitors of the PI3K/Akt/mTOR pathway are being developed, e.g., as listed in Steelman et al., Expert Opin. Ther. Targets 8:1, 2004 and Granville et al., Clin. Cancer Res. 12:679, 2006, which are herein incorporated by reference. These include inhibitors of PI3K, PDK-1 (which contributes to the activation of Akt by phosphorylating it, so should be viewed as part of the PI3/Akt pathway), Akt, and mTOR. The mTOR inhibitors include rapamycin, which has already been approved for immunosuppression but is now being tested for cancer, CCI-779, RAD-001 and AP23573, some of which have shown promising results in clinical trials and are being further tested (Granville et al., op. cit.).

### SUMMARY OF THE INVENTION

The invention provides a method of treating cancer by administering to a patient in need of such treatment a first agent that inhibits Hepatocyte Growth Factor (HGF) in combination with a second agent that is an agonist of PTEN. In a preferred embodiment, the first agent is a monoclonal antibody (mAb) that binds to and neutralizes HGF. Chimeric, human and humanized anti-HGF mAbs are especially preferred, particularly humanized L2G7. The second agent is a PTEN agonist, that is an agent that stimulates the expression or activity of PTEN or which substitutes for one or more of the functions of PTEN. In a preferred embodiment, the PTEN agonist is an inhibitor of mTOR, with rapamycin and CCI-779 especially preferred. The method is especially preferred for treating brain cancers such as glioma, especially glioblastoma.

### BRIEF DESCRIPTION OF THE DRAWINGS

FIG. 1. A and B. PTEN was expressed in PTEN-null U87 and A172 glioblastoma cells via adenovirus (Ad-PTEN) infections prior to treatment with HGF. The effect of PTEN expression on cell proliferation and G1/S cell cycle progression were assessed by cell counting (A) and by propidium iodide flow cytometry (B), respectively. C. PTEN expression was inhibited in wild-type PTEN SF767 cells by siRNA transfection prior to treatment with HGF. Cell proliferation and cell cycle progression were analyzed as described above. The immunoblots shown confirm PTEN protein restoration in U87 and A172 and PTEN protein inhibition in SF767 cells. Ad-con=Control adenovirus.

FIG. 2. PTEN was expressed in PTEN-null U87 and A172 glioblastoma cells via adenovirus (Ad-PTEN) infections prior to treatment with HGF. The effect of PTEN expression on HGF-induced cell invasion and cell migration were analyzed using a transwell invasion assay and a migration assay. A. PTEN expression inhibited basal and HGF-induced cell invasion in U87 and A172 cells. Photographs show representative stained membranes. Graphs show quantification of invading cells (n=3). B. Photographs of migrating U87 cells show that PTEN expression inhibits basal and HGF-induced tumor cell migration.

FIG. 3. PTEN-null U87 and A172 cells were infected with adenoviruses encoding PTEN (Ad-PTEN) or control (Ad-control) prior to treatment with HGF. A. Total protein was electrophoretically separated and immunoblotted for phospho-tyrosine to determine the effects of PTEN on HGF-induced overall protein tyrosine phosphorylation. B and C. Cell lysates were immunoblotted for selected signal transduction proteins known to mediate c-Met functions.

FIG. 4. A. U87 cells were infected with adenoviruses encoding anti-HGF and anti-c-Met U1/ribozymes (Ad-U1/ribozymes), PTEN (Ad-PTEN), a combination of both, or control (Ad-con). Cell proliferation and cell cycle were assessed by cell counting and by propidium iodide flow cytometry, respectively. B. U87 and A172 cells were treated with anti-HGF monoclonal antibody (L2G7), the mTOR inhibitor rapamycin, a combination of both or control and assessed for cell proliferation as described above.

FIG. 5. PTEN-expressing U87 clones were generated by stable transfection with plasmids encoding PTEN. Two PTEN expressing clones and two control clones (3×10⁵ cells) were implanted in immunodeficient mice brains (n=10) one week prior to treatment with anti-HGF antibody L2G7 twice per week for three weeks. The animals were euthanized one week after the last treatment and tumor cross-sectional areas were measured on H&E stained brain cross sections with computer-assisted image analysis. Immunoblots at upper right show PTEN levels in control and PTEN-expressing clones. Representative brain tumor cross-sections are shown in the lower panel and quantification of tumor cross-sectional areas is shown in the upper panel.

FIG. 6. Wild-type U87 glioblastoma cells (3×10⁵) were implanted stereotactically in immunodeficient mice brains (n=10). One week post-tumor implantation, the animals were treated with i.p. injections of L2G7 (100 μg twice per week), the mTOR inhibitor rapamycin (40 μg three times per week), L2G7+rapamycin (same dose schedule as when used individually), or vehicle control for 2 weeks. The animals were euthanized 1 week after the last treatment and tumor cross-sectional areas were measured on H&E-stained brain cross-sections with computer-assisted image analysis. Upper panel, quantification of tumor cross-sectional areas; lower panel, representative brain tumor cross-sections. *, P<0.05, relative to control; #, P<0.05, relative to single treatment; bars, SE.

FIG. 7. Amino acid sequences of the entire HuL2G7 heavy chain (A) (SEQ ID NO:1) and light chain (B) (SEQ ID NO:2). The first amino acids of the mature heavy and light chain V regions (i.e., after cleavage of the signal sequences) are double underlined and labeled with the number 1; these amino acids are therefore the first amino acids of the light and heavy chains of the actual HuL2G7 mAb. In the heavy chain, the first amino acids of the CH1, hinge, CH2 and CH3 regions are underlined, and in the light chain, the first amino acid of the C_{κ} region is underlined.

FIG. 8. Amino acid sequences of the light chain (A) (SEQ ID NO:3) and heavy chain (B) (SEQ ID NO:4) variable regions of the 2.12.1 human monoclonal antibody disclosed in WO 2005/017107 A2, therein designated respectively as SEQ ID NOS. 38 and 39. The first amino acids of the mature heavy and light variable regions (i.e., after cleavage of the signal sequences), and thus of the actual 2.12.1 mAb, are double underlined.

FIG. 9. Combination effects of HuL2G7 and Rapamycin in glioblastoma U-87 MG xenograft model. Data represent the mean±SD (n=6). *p≦0.025 by one-tailed Steel test (v.s. vehicle).

FIG. 10. Comparison of differential tumor volumes of the mice group treated with HuL2G7, Rapamycin, and a combination of the two. Data represent the mean±SD (n=6). ^{#} p≦0.05, ^{###} p≦0.001 by t-test (vs. combination).

### DETAILED DESCRIPTION OF THE PREFERRED EMBODIMENTS

The invention provides a method of treating cancer by administering to a patient in need of such treatment a first agent that inhibits the activity of Hepatocyte Growth Factor (HGF), i.e., an HGF antagonist or cMet antagonist, in combination with (i.e., together with) a second agent that is an agonist of PTEN. In many embodiments, the first agent is a monoclonal antibody (mAb) and/or the second agent is an mTOR inhibitor.

### 1. Antibodies

Antibodies are very large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-D space to form the actual antibody binding site which locks onto the target antigen. The position and length of the CDRs have been precisely defined. Kabat, E. et al., Sequences of Proteins of Immunological Interest, U.S. Department of Health and Human Services, 1983, 1987. The part of a variable region not contained in the CDRs is called the framework, which forms the environment for the CDRs.

A monoclonal antibody (mAb) is a single molecular species of antibody and therefore does not encompass polyclonal antibodies produced by injecting an animal (such as a rodent, rabbit or goat) with an antigen, and extracting serum from the animal. A humanized antibody is a genetically engineered monoclonal antibody in which the CDRs from a mouse antibody (“donor antibody”, which can also be rat, hamster or other similar species) are grafted onto a human antibody (“acceptor antibody”). Humanized antibodies can also be made with less than the complete CDRs from a mouse antibody (e.g., Pascalis et al., J. Immunol. 169:3076, 2002). Thus, a humanized antibody is an antibody having CDRs from a donor antibody and variable region frameworks and constant regions from human antibodies. The light and heavy chain acceptor frameworks may be from the same or different human antibodies and may each be a composite of two or more human antibody frameworks; or alternatively may be a consensus sequence of a set of human frameworks (e.g., a subgroup of human antibodies as defined in Kabat et al., op. cit.), i.e., a sequence having the most commonly occurring amino acid in the set at each position. In addition, in order to retain high binding affinity, at least one of two additional structural elements can be employed. See, U.S. Pat. Nos. 5,530,101 and 5,585,089, each of which is incorporated herein by reference, which provide detailed instructions for construction of humanized antibodies.

In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. Sequence identity is determined when antibody sequences being compared are aligned according to the Kabat numbering convention. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

As used herein, the term “human-like” antibody refers to a mAb in which a substantial portion of the amino acid sequence of one or both chains (e.g., about 50% or more) originates from human immunoglobulin genes. Hence, human-like antibodies encompass but are not limited to chimeric, humanized and human antibodies. As used herein, a “reduced-immunogenicity” antibody is one expected to have significantly less immunogenicity than a mouse antibody when administered to human patients. Such antibodies encompass chimeric, humanized and human antibodies as well as antibodies made by replacing specific amino acids in mouse antibodies that may contribute to B- or T-cell epitopes, for example exposed residues (Padlan, Mol. Immunol. 28:489, 1991). As used herein, a “genetically engineered” antibody is one for which the genes have been constructed or put in an unnatural environment (e.g., human genes in a mouse or on a bacteriophage) with the help of recombinant DNA techniques, and would therefore, e.g., not encompass a mouse mAb made with conventional hybridoma technology.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

### 2. Antibodies for Use in the Invention

A monoclonal antibody (mAb) that binds HGF (i.e., an anti-HGF mAb) is said to neutralize HGF, or be neutralizing, if the binding partially or completely inhibits one or more biological activities of HGF (i.e., when the mAb is used as a single agent). Among the biological properties of HGF that a neutralizing antibody may inhibit are the ability of HGF to bind to its cMet receptor, to cause the scattering of certain cell lines such as Madin-Darby canine kidney (MDCK) cells; to stimulate proliferation of (i.e., be mitogenic for) certain cells including hepatocytes, Mv 1 Lu mink lung epithelial cells, and various human tumor cells; or to stimulate angiogenesis, for example as measured by stimulation of human vascular endothelial cell (HUVEC) proliferation or tube formation or by induction of blood vessels when applied to the chick embryo chorioallantoic membrane (CAM). Antibodies for use in the invention preferably bind to human HGF, i.e., to the protein encoded by the GenBank sequence with Accession number D90334.

A neutralizing anti-HGF mAb is preferred for use as the first agent in the invention and, at a concentration of, e.g., 0.01, 0.1, 0.5, 1, 2, 5, 10, 20 or 50 μg/ml, inhibits a biological function of HGF (e.g., stimulation of proliferation or scattering) by about at least 50% but preferably 75%, more preferably by 90% or 95% or even 99%, and most preferably approximately 100% (essentially completely) as assayed by methods known in the art. Inhibition is considered complete if the level of activity is within the margin of error for a negative control lacking HGF. Typically, the extent of inhibition is measured when the amount of HGF used is just sufficient to fully stimulate the biological activity, or is 0.05, 0.1, 0.5, 1, 3 or 10 μg/ml. Preferably, at least 50%, 75%, 90%, or 95% or essentially complete inhibition is achieved when the molar ratio of antibody to HGF is 0.5×, 1×, 2×, 3×, 5× or 10×. Preferably, the mAb is neutralizing, i.e., inhibits the biological activity, when used as a single agent, but optionally 2 mAbs can be used together to give inhibition. Most preferably, the mAb neutralizes not just one but several of the biological activities listed above; for purposes herein, an anti-HGF mAb that used as a single agent neutralizes all the biological activities of HGF is called “fully neutralizing”, and such mAbs are most preferable. Anti-HGF mAbs for use in the invention are preferably specific for HGF, that is they do not bind, or only bind to a much lesser extent (e.g., Ka at least ten-fold less), proteins that are related to HGF such as fibroblast growth factor (FGF) and vascular endothelial growth factor (VEGF). Preferred antibodies lack agonistic activity toward HGF. That is, the antibodies block interaction of HGH with cMet without stimulating cells bearing HGF directly. Anti-HGF mAbs for use in the invention typically have a binding affinity (Kₐ) for HGF of at least 10⁷ M⁻¹ but preferably 10⁸ M⁻¹ or higher, and most preferably 10⁹ M⁻¹ or higher or even 10¹⁰ M⁻¹ or higher.

MAbs for use in the invention include antibodies in their natural tetrameric form (2 light chains and 2 heavy chains) and may be of any of the known isotypes IgG, IgA, IgM, IgD and IgE and their subtypes, i.e., human IgG1, IgG2, IgG3, IgG4 and mouse IgG1, IgG2a, IgG2b, and IgG3. The mAbs are also meant to include fragments of antibodies such as Fv, Fab and F(ab′)₂; bifunctional hybrid antibodies (e.g., Lanzavecchia et al., Eur. J. Immunol. 17:105, 1987), single-chain antibodies (Huston et al., Proc. Natl. Acad. Sci. USA 85:5879, 1988; Bird et al., Science 242:423, 1988); single-arm antibodies (Nguyen et al., Cancer Gene Ther. 10:840, 2003); and antibodies with altered constant regions (e.g., U.S. Pat. No. 5,624,821). The mAbs may be of animal (e.g., mouse, rat, hamster or chicken) origin, or they may be genetically engineered. Rodent mAbs are made by standard methods well-known in the art, comprising multiple immunization with HGF in appropriate adjuvant i.p., i.v., or into the footpad, followed by extraction of spleen or lymph node cells and fusion with a suitable immortalized cell line, and then selection for hybridomas that produce antibody binding to HGF, e.g., see under Examples. Chimeric and humanized mAbs, made by art-known methods mentioned supra, are preferred for use in the invention. Human antibodies made, e.g., by phage display or transgenic mice methods are also preferred (see e.g., Dower et al., McCafferty et al., Winter, Lonberg et al., Kucherlapati, supra). More generally, human-like, reduced immunogenicity and genetically engineered antibodies as defined herein are all preferred.

The neutralizing anti-HGF mAb L2G7 (which is produced by a hybridoma deposited at the American Type Culture Collection under ATCC Number PTA-5162 according to the Budapest treaty) as described in Kim et al., Clin Cancer Res 12:1292, 2006 and U.S. Pat. No. 7,220,410 and particularly its chimeric and humanized foams such as HuL2G7, as described in WO 07115049 A2, are especially preferred as the first agent in the invention. Neutralizing mAbs with the same or overlapping epitope as L2G7 and/or that compete with L2G7 for binding to HGF are also preferred. MAbs that are 90%, 95% or 99% identical to L2G7 in amino acid sequence, when aligned according to the Kabat numbering convention, at least in the CDRs, and maintain its functional properties, or which differ from it by a small number of functionally inconsequential amino acid substitutions (e.g., conservative substitutions), deletions, or insertions can also be used in the invention.

Also preferred for use as the first agent in the invention are the anti-HGF mAbs described in WO 2005/017107 A2, whether explicitly by name or sequence or implicitly by description or relation to explicitly described mAbs. Especially preferred mAbs are those produced by the hybridomas designated therein as 1.24.1, 1.29.1, 1.60.1, 1.61.3, 1.74.3, 1.75.1, 2.4.4, 2.12.1, 2.40.1 and 3.10.1, and respectively defined by their heavy and light chain variable region sequences provided by SEQ ID NO's 24-43, with 2.12.1 being most preferred; mAbs possessing the same respective CDRs as any of these listed mAbs; mAbs having light and heavy chain variable regions that are at least 90%, 95% or 99% identical to the respective variable regions of these listed mAbs or differing from them only by inconsequential amino acid substitutions, deletion or insertions; mAbs binding to the same epitope of HGF as any of these listed mAbs, and all mAbs encompassed by claims 1 through 94 therein.

Alternatively, any of the HGF binding proteins described in WO07143090A2 or WO07143098A2 may be used as the first agent in the invention.

Native mAbs for use in the invention may be produced from their hybridomas. Genetically engineered mAbs, e.g., chimeric or humanized mAbs, may be expressed by a variety of art-known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs for use in the invention may be purified according to standard procedures of the art such as microfiltration, ultrafiltration, protein A or G affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses. The mAbs are typically provided in a pharmaceutical formulation, i.e., in a physiologically acceptable carrier, optionally with excipients or stabilizers. Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16^{th} edition, Osol, A. Ed. 1980). The mAb is typically present at a concentration of 1-100 mg/ml, e.g., 10 mg/ml.

### 3. Other Agents for Use in the Invention

Besides anti-HGF mAbs, the first agent for use in the invention may be any other agent that inhibits HGF, i.e., inhibits its biological activity, and may therefore be called an HGF antagonist. Examples are soluble forms of cMet (e.g., see Michieli et al., Cancer Cell 6:61, 2004) and a cocktail of several anti-HGF mAbs (Cao et al., Proc. Natl. Acad. Sci. USA 98:7443, 2001). As used herein the term “agent that inhibits HGF” or “HGF inhibitor” also includes an agent that interacts with the cMet receptor of HGF so as to inhibit HGF signaling through cMet; such an agent may also be called a cMet inhibitor or antagonist. However, as used herein, inhibitors or antagonists of HGF or cMet or the HGF/cMet pathway are not meant to include agents that inhibit signaling events, such as activation of MAP kinase, that occur after (i.e., downstream) of the HGF-cMet interaction and activation of cMet, and which the HGF/cMet pathway shares with other ligand/receptor systems. A cMet antagonist may function by binding to cMet and competitively blocking binding of HGF or activation by HGF. Exemplary agents include truncated HGF proteins such as NK1, NK2, and NK4 (supra) and anti-cMet mAbs. A preferred example is an anti-cMet antibody that has been genetically engineered to have only one “arm”, i.e. binding domain, such as OA-5D5 (Martens et al., Clin. Cancer Res. 12:6144, 2006). Such agents may also be small molecule inhibitors of the tyrosine kinase activity of cMet including SU5416 (Wang et al., J Hepatology 41:267, 2004), and ARQ 197 being developed by ArQule, Inc. (Abstract Number 3525 at the 2007 Annual Meeting of the American Society of Clinical Oncology), which may be administered orally.

The second agent for use in the invention is any agonist of PTEN, preferably human PTEN. As used herein, an “agonist of PTEN” or “PTEN agonist” means an agent that stimulates the expression of PTEN in a cell, or stimulates the activity of PTEN, or which can provide one or more of the functions of PTEN, e.g., in regulating the PI3K/Akt/mTOR pathway. For example, PTEN is able to indirectly reduce the activity of mTOR (mammalian target of rapamycin) by downregulating the activity of Akt An inhibitor of mTOR directly reproduces this particular role of PTEN—reduction of mTOR activity—so such an inhibitor is considered herein to be a PTEN agonist. This type of PTEN agonist will replace some but not necessarily all the functions of the tumor suppressor PTEN in a cancer cell with mutated or deleted PTEN, and may therefore cause the cell to revert to a more normal, less malignant phenotype. Preferred PTEN agonists/mTOR inhibitors for use in the invention include rapamycin (Rapamune®, sirolimus, ATC code L04AA10 commercially available from Wyeth) and its chemical analogues such as CCI-779 (temsirolimus, Anatomical Therapeutic Chemical (ATC) code L01XE09, commercially available from Wyeth), RAD-001 (everolimus, ATC code L04AA18. commercially available from Novartis) and AP-2357 (Granville et al., op. cit.). Many PTEN agonists are small molecules (i.e., a compound having relatively low molecular weight, most often less than 500 or 600 kDa, or about 1000 kDa in the case of a macrolide such as rapamycin). Other agonists include mAbs, and zinc finger proteins or nucleic acids encoding the same, engineered to bind to and activate transcription of PTEN (see, e.g., WO 00/00388). Other PTEN agonists are described in US20070280918. Whereas proteins are typically administered parenterally, e.g. intravenously, small molecules may be administered parenterally or orally. PTEN and mTOR (also known as FRAP1) are well known human proteins for which sequences are available from UniProtKB/Swiss-Prot and similar databases. Insofar as a protein has more than one known form in a species due to natural allelic variation between individuals, an inhibitor can bind to and inhibit any, or all, of such known allelic forms, and preferably binds to and inhibits the wildtype, most common or first published allelic form. Exemplary sequences for human PTEN and mTOR(FRAP1) are assigned UniProtKB/Swiss-Prot accession numbers P60484 and P42345.

### 4. Treatment Methods

The invention provides methods of treatment in which the indicated first and second agents are administered to patients having a cancer (therapeutic treatment) or at risk of occurrence or recurrence of cancer (prophylactic treatment). The term “patient” includes human patients; veterinary patients, such as cats, dogs and horses; farm animals, such as cattle, sheep, and pigs; and laboratory animals used for testing purposes, such as mice and rats. The methods are particularly amenable to treatment of human patients. In some methods, the patient has a tumor including cells with reduced PTEN expression or activity relative to cells of noncancerous tissue of the same type, optionally from the same patient Such reduced expression or activity can be measured at the level of DNA (inferred from presence of a mutation), mRNA or protein. The mAb or other agent used in methods of treating human patients binds to the respective human protein. A mAb or other agent to a human protein can also be used in other species in which the species homolog has antigenic crossreactivity with the human protein. In species lacking such crossreactivity, an antibody or other agent is used with appropriate specificity for the species homolog present in that species. However, in xenograft experiments in laboratory animals, a mAb or other agent with specificity for the human protein expressed by the xenograft is generally used.

A mAb or other protein used as the first agent in the methods of the invention can be administered to a patient by any suitable route, especially parentally by intravenous (IV) infusion or bolus injection, intramuscularly or subcutaneously or intraperitoneally. IV infusion can be given over as little as 15 minutes, but more often for 30 minutes, 60 minutes, 90 minutes or even 2 or 3 hours. The agent can also be injected directly into the site of disease (e.g., the tumor itself; or the brain or its surrounding membranes or cerebrospinal fluid in the case of a brain tumor) or encapsulated into carrying agents such as liposomes. However, when treating brain tumors (i.e., a tumor existing within the brain of a patient), systemic administration of the mAb, e.g., by IV infusion, is possible and even preferred (see WO 06130773 A2). The dose given to a patient having a cancer is sufficient to alleviate or at least partially arrest the disease being treated (“therapeutically effective dose”) and is sometimes 0.1 to 5 mg/kg body weight, for example 1, 2, 3, 4, 5 or 6 mg/kg, but may be as high as 10 mg/kg or even 15 or 20 or 30 mg/kg. A fixed unit dose may also be given, for example, 50, 100, 200, 500 or 1000 mg, or the dose may be based on the patient's surface area, e.g., 100 mg/m². Usually between 1 and 8 doses, (e.g., 1, 2, 3, 4, 5, 6, 7 or 8) are administered to treat cancer, but 10, 12, 20 or more doses may be given. The agent can be administered daily, biweekly, weekly, every other week, monthly or at some other interval, depending, e.g. on its half-life, for 1 week, 2 weeks, 4 weeks, 8 weeks, 3-6 months or longer, or until the disease progresses. Repeated courses of treatment are also possible, as is chronic administration.

When a small molecule is used as the first or second agent, it is typically administered more often, preferably once a day, but 2, 3, 4 or more times per day is also possible, as is every two days, weekly or at some other interval. Small molecule drugs are often taken orally but parenteral administration is also possible, e.g., by IV infusion or bolus injection or subcutaneously or intramuscularly. Doses of small molecule drugs are typically 1 or 10 to 1000 mg, with 100, 150, 200 or 250 mg very typical, with the optimal dose established in clinical trials. For either a protein or small molecule drug, a regime of a dosage and intervals of administration that alleviates or at least partially arrests the symptoms of a disease (biochemical, histologic and/or clinical), including its complications and intermediate pathological phenotypes in development of the disease is referred to as a therapeutically effective regime.

When a first agent (an HGF inhibitor) is used in combination with a second agent (a PTEN agonist, e.g., an mTOR inhibitor), the combination may take place over any convenient timeframe. For example, each agent may be administered to a patient on the same day, and the agents may even be administered in the same intravenous infusion. However, the agents may also be administered on alternating days or alternating weeks, fortnights or months, and so on. In some methods, the respective agents are administered with sufficient proximity in time that the agents are simultaneously present (e.g., in the serum) at detectable levels in the patient being treated. In some methods, an entire course of treatment of one agent consisting of a number of doses over a time period (see above) is followed by a course of treatment of the other agent also consisting of a number of doses. In some methods, treatment with the agent administered second is begun if the patient has resistance or develops resistance to the agent administered initially. The patient may receive only a single course of treatment with each agent or multiple courses with one or both agents. Frequently, a recovery period of 1, 2 or several days or weeks is allowed between administration of the two agents if this is beneficial to the patient in the judgment of the attending physician. When a suitable treatment regiment has already been established for one of the agents, that regimen is preferably used when the agent in used in combination with the other. Typically, these agents are administered until the disease progresses.

The methods of the invention can also be used in prophylaxis of a patient at risk of cancer. Such patients include those having genetic susceptibility to cancer, patients who have undergone exposure to carcinogenic agents, such as radiation or toxins, and patients who have undergone previous treatment for cancer and are at risk of recurrence. A prophylactic dosage is an amount sufficient to eliminate or reduce the risk, lessen the severity, or delay the outset of the disease, including biochemical, histologic and/or clinical symptoms of the disease, its complications and intermediate pathological phenotypes presenting during development of the disease. Administration of a pharmaceutical composition in an amount and at intervals effective to effect one or more of these objects is referred to as a prophylactically effective regime. The dosages and regimens disclosed above for therapeutic treatment can also be used for prophylactic treatment.

Types of cancer especially susceptible to treatment using the methods of the invention include solid tumors known or suspected to require angiogenesis or to be associated with elevated levels of HGF or cMet (which can be measured at the mRNA or protein level relative to noncancerous tissue of the same type, optionally from the same patient), for example ovarian cancer, breast cancer, lung cancer (small cell or non-small cell), colon cancer, prostate cancer, pancreatic cancer, bladder cancer, cervical cancer, renal cancer, gastric cancer, liver cancer, head and neck tumors, mesothelioma, melanoma, and sarcomas, and brain tumors. Treatment can also be administered to patients having leukemias or lymphomas. The methods of the invention are particularly suitable for treatment of brain tumors including meningiomas; especially gliomas including ependymomas, oligodendrogliomas, and all types of astrcytomas (low grade, anaplastic, and glioblastoma multiforme or simply glioblastoma); gangliogliomas, schwannomas, chordomas; and brain tumors primarily of children, particularly medulloblastoma but also including primitive neuroectodermal tumors. Both primary brain tumors (i.e., arising in the brain) and secondary or metastatic brain tumors can be treated by the methods of the invention. Tumors associated with loss of PTEN function such as melanoma, endometrial, breast, prostate, ovarian, lung, bladder, gastric, cervix, head and neck, and renal are also especially susceptible to treatment by the methods of the invention.

Because of the severity of cancer, several drugs to treat the disease are often given in combination. Hence, in a preferred embodiment of the present invention, the first agent (an HGF inhibitor) and the second agent (a PTEN agonist, e.g., an mTOR inhibitor) are administered together with additional anti-cancer drugs. The first agent and second agent can be administered before, during or after the other anti-cancer drugs. For example, the first and second agents may be administered together with any one or more of the chemotherapeutic drugs known to those of skill in the art of oncology, for example alkylating agents such as carmustine, chlorambucil, cisplatin, carboplatin, oxaliplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere®; the topoisomerase 1 inhibitor irinotecan; agents specifically approved for brain tumors including temozolomide and Gliadel® wafer containing carmustine; and inhibitors of tyrosine kinases such as Gleevec®, Sutent® (sunitinib malate) and Tarceva® (erlotinib); and all approved and experimental anti-cancer agents listed in WO 2005/017107 A2 (which is herein incorporated by reference). The first and second agents can be administered in combination with 1, 2, 3 or more of these other agents used in a standard chemotherapeutic regimen. Normally, the other agents are those already known to be effective for the particular type of cancer being treated. Moreover, the first and second agents can be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon), and/or with surgery. Combination with radiation therapy can be especially appropriate for head and neck cancer and brain tumors. Other agents with which the first and second agents can be administered include biologics such as monoclonal antibodies, including Herceptin™ against the HER2 antigen, Avastin™ against VEGF, and Erbitux® (cetuximab) and Vectibix® (panitumumab) against the Epidermal Growth Factor (EGF) receptor (EGFR).

Optionally, an HGF inhibitor and a PTEN agonist can be combined in a combination product or kit, for example, as separate vials in the same package, or holder. Some combinations of an HGF inhibitor and a PTEN agonist (e.g., two antibodies) can also be mixed in the same composition. Such compositions and kits can be formed either by a manufacturer or by a health care provider. Kits and compositions can be provided with instructions for use in any of the methods of the invention.

The progression-free survival or overall survival time of patients with cancer (e.g., ovarian, prostate, breast, lung, colon, pancreatic, kidney, and brain, especially when relapsed or refractory) treated according to the method of the invention with the first and second agents may increase by at least 10%, 20%, 30% or 40% but preferably 50%, 60% to 70% or even 80%, 90%, 100% or longer, compared to patients treated similarly (e.g., with standard chemotherapy or without specific therapy) but without the first and second agents. The median progression-free survival or overall survival time may also be increased by at least 10 days, but preferably 30 days, 60 days, or 3, 4, 5 or 6 months or 1 year or longer by treatment according to the method of the invention. In addition or alternatively, treatment by the method of the invention may increase the complete response rate, partial response rate, or objective response rate (complete+partial) of patients by at least 10%, 20%, 30% or 40% but preferably 50%, 60% to 70% or even 80%, 90% or 100%. Moreover, when administering treatment with two agents, the regimes with which the respective agents are administered are combined in such a manner that each agent can make a contribution to the therapy, so treatment according to the invention with the first and second agents can increase progression-free or overall survival or increase the complete, partial or objective response rate by at least 10%, 20%, 30% or 40% but preferably 50%, 60% to 70% or even 80%, 90% or 100% compared to treatment with either agent without the other. Indeed, preferably treatment with the first and second agents is synergistic, i.e., better than additive. Optionally, treatment according to the method of the invention can inhibit tumor invasion, or metastasis.

Typically, in a clinical trial (e.g., a phase II, phase II/III or phase III trial), the aforementioned increases in median progression-free survival and/or response rate of the patients treated by the method of the invention together with a standard therapy (e.g., a chemotherapeutic regimen), relative to the control group of patients receiving the standard therapy alone, is statistically significant, for example at the p<0.05 or 0.01 or even 0.001 level. The complete and partial response rates can be determined by objective criteria commonly used in clinical trials for cancer, e.g., as listed or accepted by the National Cancer Institute and/or Food and Drug Administration.

### EXAMPLES

### 1. Materials and Methods Used

Cell culture and reagents. Human U87 glioblastoma cells were grown in Eagle's Minimum Essential Medium (Cellgro Mediatech, Washington, D.C.) containing 10% fetal bovine serum, 0.15% sodium bicarbonate, 1 mmol/L sodium pyruvate, 0.1 mol/L and nonessential amino acids (Life Technologies, Rockville, Md.). Human A172 glioblastoma cells were grown in Dulbecco's Minimum Essential Medium (Cellgro Mediatech, Washington, D.C.) supplemented with 10% Fetal Bovine Serum and 4.5 g/L glucose. Human SF767 glioblastoma cells (a kind gift from Dr. Russel Pieper, UCSF) were grown in Minimum Essential Medium (Invitrogen, Carlsbad, Calif.) supplemented with 10% fetal calf serum. All cells were grown at 37° C. in 5% CO₂-95% O₂. The mTOR inhibitor rapamycin was purchased from Sigma-Aldrich (St. Louis, Mo.). PTEN and control siRNA were purchased from Santa Cruz, (Santa Cruz, Calif.). Human recombinant HGF was used.

Vectors and transfections. Adenoviruses encoding wild-type PTEN (Ad-PTEN), anti-HGF U1/ribozymes (Ad-U1/HGF), anti-c-Met U1/ribozymes (Ad-U1/Met), and control adenovirus (Ad-control) were constructed as previously described (Abounader et al., Faseb J 16:108, 2002; Abounader et al., Oncogene 23:9173, 2004). The cells were infected with adenovirus vectors (MOI=10) for 24 hrs prior to treatment with 50 ng/ml HGF. For stable PTEN expression, the full-length human PTEN cDNA was subcloned into the mammalian expression vector pcDNA3.1-Zeo (Invitrogen, Carlsbad, Calif.) to generate pcDNA/PTEN. Cells were transfected with pcDNA/PTEN using Fugene transfection reagent (Roche Applied Science, Indianapolis, Ind.) according to the manufacturer's instructions. Transfected cells were selected in zeocin and PTEN expression was verified by immunoblotting.

siRNA knock-down. PTEN expression was inhibited in SF767 cells expressing wild-type PTEN with PTEN siRNA (Santa Cruz, Santa Cruz, Calif.). The cells were transfected with 10 nM PTEN siRNA or scrambled control siRNA using oligofectamine transfection reagent (Invitrogen, Carlsbad, Calif.) according to the manufacturer's instructions. PTEN protein inhibition was verified by immunoblotting.

Growth curves. U87 cells (30,000)/well or A172 cells (40,000)/well were seeded in 10% FBS medium, transferred to 0.1% FBS medium (for c-Met activation experiments) or 3% FBS medium (for c-Met inhibition experiments) and infected with adenoviruses for 24 hrs prior to treatment with 50 ng/ml HGF. The cells were harvested by trypsinization every day for five days and counted with a hemocytometer. To assess effects of combining the mTOR inhibitor rapamycin with anti-HGF antibody (L2G7) on cell proliferation, U87 cells (30,000/well) or A172 cells (40,000/well) were grown in 10% FBS medium, exposed to 0.1% FBS medium and treated with 40 nM rapamycin and/or 20 μg/mL L2G7. The cells were counted as described above.

Cell cycle analysis. The effects of c-Met and PTEN on cell cycle progression were assessed using propidium iodide (PI) flow cytometry as previously described (Li et al. Cancer Res. 65:9355, 2005). Briefly, U87, A172 or SF767 cells were plated in 10% FBS medium overnight, and subsequently exposed to 0.1% FBS (for c-Met activation experiments) or 3% FBS medium (for c-Met inhibition experiments) and infected with adenoviruses or transfected with siRNA for 24 hrs prior to treatment with or without 50 ng/ml HGF for additional 24 hrs. The cells were washed with PBS, harvested and fixed in 70% (v/v) ethanol. The cells were then treated with 20 μg of DNase-free RNase and stained with propidium iodide. Cell samples were analyzed on a FACscan (Becton-Dickinson, Fullerton, Calif.).

Invasion assay. The effects of c-Met and PTEN on cell invasion were assessed using a transwell invasion assay (BD Biosciences, San Jose, Calif.). A cell culture insert was coated with 30 μl collagen type IV (250 μg/mL). U87 or A172 cells (1×10⁵) were infected with adenoviruses for 24 hrs, resuspended in 300 μL 0.1% FBS medium with or without 50 ng/ml HGF and placed in the upper chamber. 600 μL 10% FBS medium were placed in the lower chamber. After incubation for 6 hrs at 37° C. in 5% CO₂, the cells on the upper membrane surface were mechanically removed. Cells that migrated to the lower side of the membrane were fixed and stained with 0.1% crystal violet (Sigma-Aldrich, St. Louis, Mo.). Photographs were taken and stained cells were counted under a microscope in five randomly chosen fields.

Migration assay. The effects of c-Met and PTEN on cell migration were assessed by a scratch wound assay. Briefly, cells grown in 6-well plates were infected with adenoviruses for 48 hrs and the medium was changed to 0.1% FBS overnight. The nearly confluent cell monolayers were then carefully scratched using 20 μL sterile pipette tips. Cell debris was removed by washing with PBS. The wounded monolayers were kept in 0.1% FBS medium with or without 50 ng/ml HGF for additional 48 hrs and photographs were taken under a light microscope.

Immunoprecipitation and immunoblotting. Immunoprecipitation was used to determine if PTEN binds to c-Met. Briefly, U87 and A172 cells were transfected with the PTEN trapping mutant D92 (a kind gift of Dr. Kenneth Yamada, NIH) or GFP (control). D92 irreversibly binds to its target and has been previously successfully used to determine direct targets of PTEN (Gu et al. J. Cell Biol. 146:389, 1999). Twenty-four hrs post-transfection, the cells were treated with 50 ng/ml HGF for 10 min and subsequently lysed with RIPA buffer (1% Igepal, 0.5% sodium deoxycholate and 0.1% SDS in PBS). One mg of protein was incubated with 4 μg c-Met antibody (Upstate Biotechnology,) or PTEN antibody (Cell signaling Technologies, Danvers, Mass.) for 2 hrs before incubation with protein A plus G beads (Santa Cruz Biotechnologies, Santa Cruz, Calif.) overnight at 4° C. The beads were collected by centrifugation, washed five times with lysis buffer, heated to 100° C. in Laemmli buffer, and subjected to immunoblotting for PTEN or c-Met, respectively as described below. Immunoblotting was performed as previously described using antibodies specific for phosphotyrosine, phospho-c-Met, phospho-MAPK, MAPK, phospho-Akt, Akt, phospho-GSK-3α/β, GSK-3α/β (Cell Signaling Technologies, Danvers, Mass.), Cdk2, cyclin E, E2F-1, PTEN, β-actin (Santa Cruz Biotechnologies, Santa Cruz, Calif.) and p27 (BD Biosciences, San Jose, Calif.) (Li et al., 2005, op. cit.; Li et al. Lab Invest. 88:98, 2008).

Tumor formation in vivo. The effects of combining PTEN restoration with HGF inhibition using anti-HGF monoclonal antibody (L2G7) treatment on in vivo tumor growth were tested in intracranial animal xenograft models. Stable clones of U87 cells expressing wild-type PTEN or control were generated as described above. Clones (3×10⁵ cells) were stereotactically implanted into the brains of immunodeficient mice. One week post tumor implantation, the animals were treated with intraperitoneal injections of 100 μL2G7 or control twice per week for three weeks. The animals were euthanized one week after the last treatment. The brains were removed, sectioned, and stained with H&E. Maximal tumor cross-sectional area was measured by computer-assisted image analysis.

Statistics. All immunoblots show representative results from at least two experiments. All other experiments were repeated at least 3 times and the results were expressed as mean±s.e.m. When appropriate, two group comparisons were analyzed with a t-test and multiple group comparisons were analyzed with a Dunnett test and p values were calculated. P<0.05 was considered significant.

### 2. The PTEN Status Affects c-Met-Dependent Glioblastoma Cell Proliferation and Cell Cycle Progression

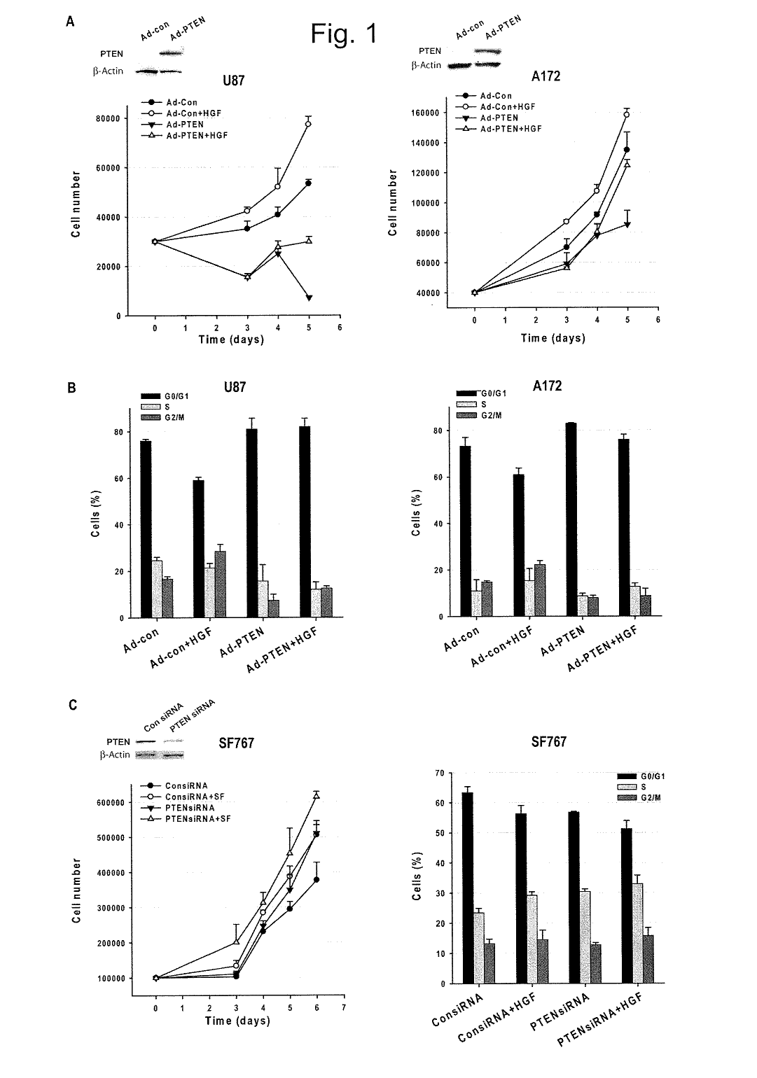

It was previously reported that c-Met activation contributes to glioblastoma malignancy by inducing cell proliferation and cell cycle progression (Abounader et al., J. Natl. Cancer Inst. 91:1548, 1999; Abounader et al., Faseb J 16:108, 2002). To determine how PTEN expression affects these c-Met-induced malignancy parameters, we restored PTEN to PTEN-null U87 and A172 glioblastoma cells and inhibited PTEN in wild-type PTEN SF767 glioblastoma cells and studied the effects of c-Met activation on cell proliferation and cell cycle progression in these settings. PTEN was restored to U87 and A172 cells by infection with Ad-PTEN or Ad-Control for 24 hrs. (MOI=10). The cells were subsequently treated with or without 50 ng/ml HGF. Cell proliferation and cell cycle were analyzed as described in Example 1. PTEN expression significantly inhibited basal and HGF-induced cell proliferation in both cell lines (n=6, p<0.05: PTEN relative to control and PTEN+HGF relative to control+HGF) (FIG. 1A). PTEN expression also significantly and completely inhibited HGF-induced cell cycle progression in U87 cells (n=3, p<0.05) and partially inhibited HGF-induced cell cycle progression in A172 cells (n=4, p=<0.05) (FIG. 1B). To assess the effects of endogenous PTEN on c-Met-dependent cell proliferation and cell cycle progression, we inhibited PTEN in wild-type PTEN SF767 cells by transfection with 10 nM PTEN siRNA for 48 hrs prior to treatment with or without 50 ng/ml HGF. Inhibition of PTEN expression in SF767 cells amplified HGF-induced cell proliferation and cell cycle progression (n=3) (FIG. 1C). These data show that the PTEN status strongly affects c-Met-dependent glioblastoma cell proliferation and cell cycle progression.

### 3. PTEN Expression Inhibits c-Met-Dependent Glioblastoma Cell Invasion and Cell Migration

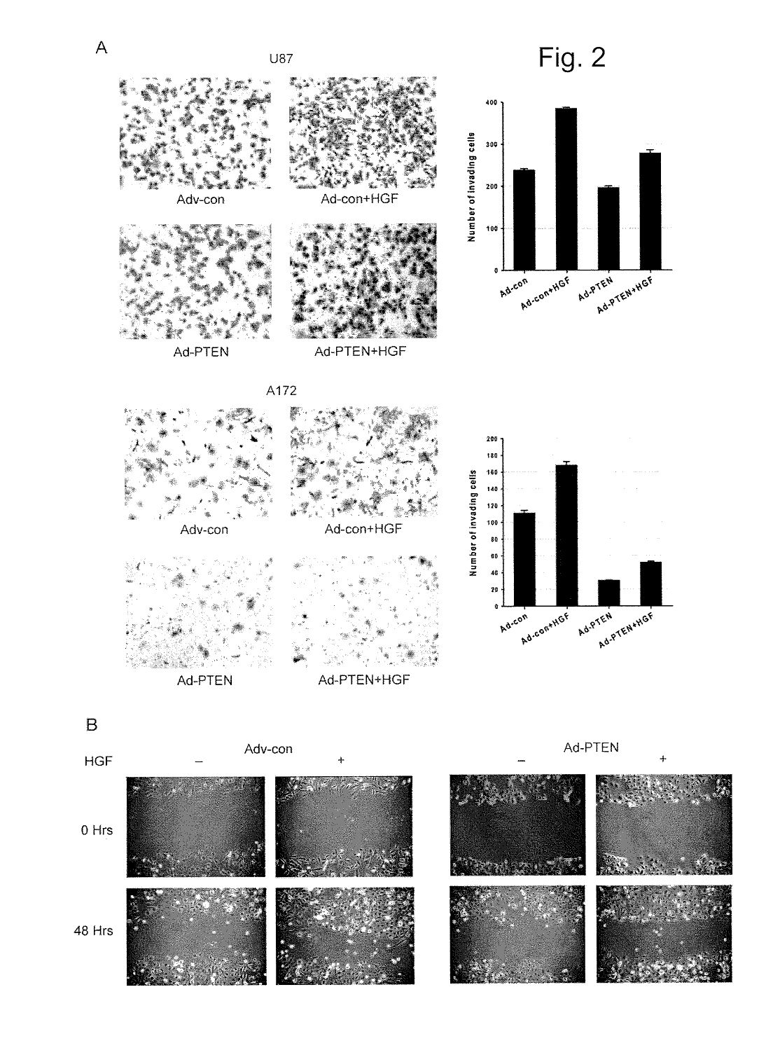

c-Met activation contributes to glioblastoma malignancy by inducing tumor cell migration and invasion (Brockmann et al., Neurosurgery 52:1391-9; discussion 9, 2003; Hamasuna et al., Int. J. Cancer 93:339, 2001). To determine if PTEN expression affects c-Met-dependent glioblastoma cell migration and invasion, we restored PTEN to PTEN-null U87 and A172 cells and assessed the effects of c-Met activation on these malignancy parameters. PTEN was restored to U87 and A172 cells by infection with Ad-PTEN or Ad-Control for 24 hrs (MOI=10). The cells were subsequently treated with or without 50 ng/ml HGF and cell invasion and migration were analyzed as described in the methods. PTEN expression significantly inhibited HGF-induced transwell cell invasion in U87 cells (n=3, p<0.05: PTEN+HGF relative to control+HGF) (FIG. 2A). PTEN expression significantly inhibited basal as well as HGF-induced transwell cell invasion in A172 cells (n=3, p<0.05: PTEN relative to control and PTEN+HGF relative to control+HGF) (FIG. 2A). PTEN expression also inhibited basal and HGF-induced cell migration in U87 cells (FIG. 2B). These data show that the PTEN status strongly affects c-Met-dependent glioblastoma cell invasion and cell migration.

### 4. PTEN Attenuates c-Met-Dependent Signal Transduction

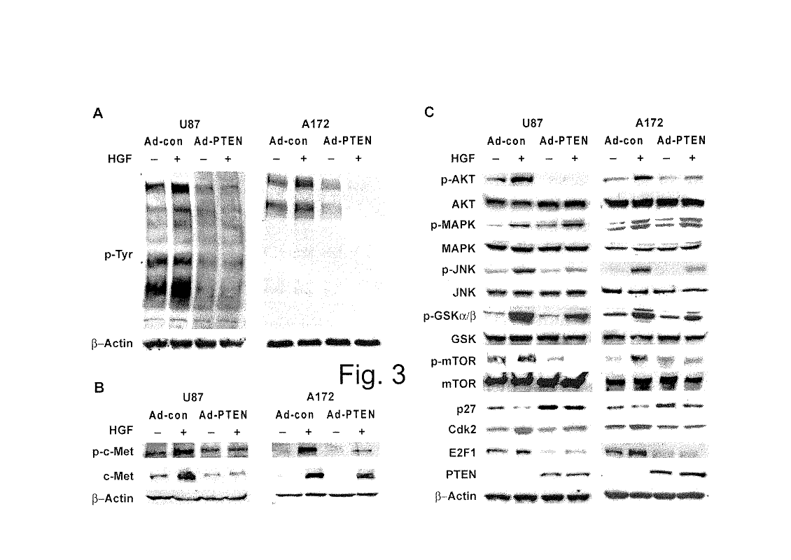

To determine how PTEN expression affects c-Met-dependent signal transduction, we restored PTEN to PTEN-null U87 and A172 cells and assessed the effects of c-Met activation on overall protein tyrosine phosphorylation and on specific signal transduction molecules previously shown to mediate c-Met functions (Abounader et al., 1999, op. cit.; Abounader et al., 2002, op. cit; Li et al., 2008, op. cit.; Abounader et al., J Neurochem 76:1497, 2001; Bowers et al., Cancer Res. 60:4277, 2000). PTEN was restored to U87 and A172 cells by infection with Ad-PTEN or Ad-Control for 24 hrs (MOI=10). The cells were subsequently treated with or without 50 ng/ml HGF for different times (5 min-24 hrs). Cell lysates were extracted and immunoblotted with anti-phosphotyrosine or with antibodies specific to the proteins being investigated. HGF treatment induced tyrosine phosphorylation of various proteins in both cell lines. PTEN restoration inhibited basal as well as HGF-induced tyrosine phosphorylation of some of these proteins (FIG. 3A). This suggests that PTEN inhibits c-Met-dependent signaling by inhibiting various molecules that are activated by HGF. To determine if PTEN directly affects c-Met phosphorylation we used immunoblotting and co-immunoprecipitation and found that PTEN does not alter c-Met phosphorylation relative to total c-Met and does not co-immunoprecipitate with c-Met (not shown). To identify some of the c-Met activated proteins that are inhibited by PTEN, the effects of PTEN and HGF on selected proteins known to mediate c-Met functions were investigated. The cells were treated as described above and analyzed for protein levels and activation with immunoblotting using specific antibodies and phospho-antibodies. Treatment of the cells with HGF markedly increased the phosphorylation of Akt, p42/44-MAPK, INK, GSK-3α/β and mTOR and induced protein levels of Cdk2 and E2F-1 and inhibited protein levels of p27. PTEN expression inhibited HGF-induced phosphorylation of AKT, JNK, GSK-3α/β and mTOR, but did not affect HGF-induced phosphorylation of c-Met and p42/44-MAPK. Consistent with its effects on the cell cycle, PTEN restoration inhibited HGF-induced E2F-1, partially inhibited HGF-induced Cdk2, and blocked HGF-induced inhibition of p27 (FIG. 3B). These data show that PTEN strongly but selectively affects c-Met-dependent signal transduction.

### 5. Combined HGF/c-Met Inhibition and PTEN Restoration or mTOR Inhibition Additively Inhibit Glioblastoma Cell Proliferation and Cell Cycle Progression

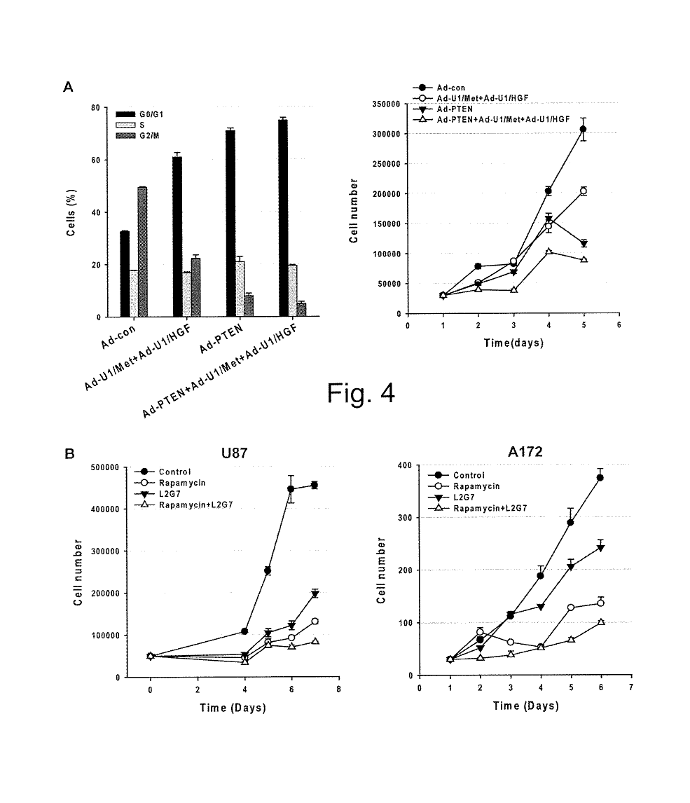

Since c-Met activation has greater malignant effects in the setting of PTEN loss, we hypothesized that combining PTEN restoration with HGF/c-Met inhibition might lead to greater anti-proliferative effects in glioblastoma. To test this hypothesis we restored PTEN and inhibited HGF/c-Met singly and in combination and assessed the effects on cell proliferation and cell cycle progression. PTEN was restored by infection of U87 cells with Ad-PTEN and HGF and c-Met were inhibited by infection of the cells with Ad-U1/HGF and Ad-U1/Met, respectively as previously described (Abounader et al., 1999, op. cit.; Abounader et al., 2002, op. cit.). PTEN expression and HGF and c-Met knock-down were verified by immunoblotting. Cell proliferation and cell cycle were assessed as described above. Single inhibition of HGF/c-Met as well as single PTEN restoration led to inhibition of cell proliferation and cell cycle progression (FIG. 4A). Combined inhibition of HGF/c-Met and PTEN restoration led to significantly greater inhibitory effects on cell cycle progression and cell proliferation as compared to single treatment (n=3, p<0.05 relative to single treatment) (FIG. 4A).

We also tested the combined effects of monoclonal anti-HGF antibody-mediated inhibition of HGF in combination with mTOR inhibition with rapamycin, since mTOR inhibition counteracts some effects of PTEN restoration and rapamycin is an approved drug. U87 and A172 cells were treated with either L2G7, rapamycin, a combination of both or control and assessed for cell proliferation. L2G7 or rapamycin single treatment significantly inhibited cell proliferation relative to control in both cell lines. Combination treatment of L2G7 and rapamycin had significantly greater inhibitory effects on tumor cell proliferation than each therapy mode alone (p<0.05, relative to single treatment). These data provide for the first time a rationale for combining anti-HGF/c-Met therapies and anti-mTOR therapies in a clinical setting.

### 6. Combining HGF Inhibition and PTEN Restoration Additively Inhibits In Vivo Glioblastoma Xenograft Growth

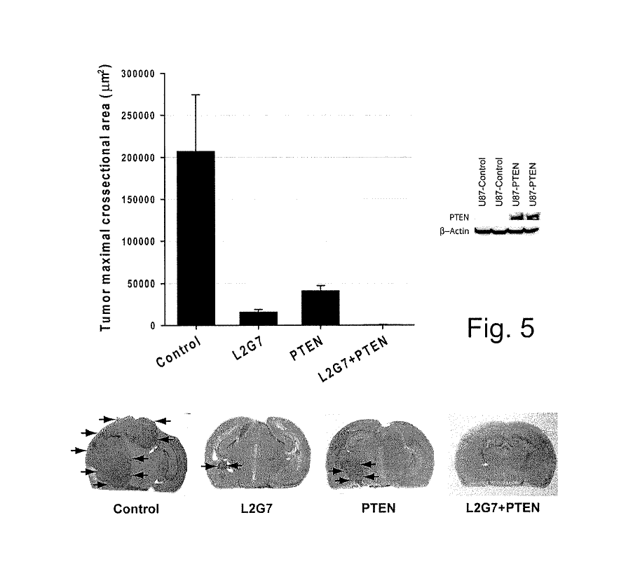

To determine if combining PTEN restoration and HGF inhibition have in vivo therapeutic advantage, we generated PTEN expressing U87 clones by stable transfection with plasmids encoding PTEN and control clones by transfection with plasmids encoding empty vector. PTEN expression was verified by immunoblotting. Two PTEN expressing clones and two control clones were implanted in immunodeficient mice one week before treatment with intraperitoneal anti-HGF antibody L2G7 or control. After 4 weeks, the animals were euthanized and tumor maximal crossectional areas were measured. Both PTEN restoration and L2G7 treatment led to a significant reduction of tumor cross sectional areas relative to control (n=10, p<0.01) (FIG. 5). The combination of PTEN restoration and L2G7 treatment displayed a significantly greater inhibition of tumor growth than each single treatment (n=10, p<0.01 relative to single treatment). Notably, in the combination group, except for one animal which developed a very small tumor, no animal showed signs of tumor growth but only scar tissue around the needle track. Therefore, combination therapy led to an additive inhibitory effect on in vivo tumor growth and tumorigenicity over single therapies. These data show that combining PTEN restoration with HGF/c-Met inhibition has therapeutic value.

### 7. HGF Inhibitors and PTEN Agonists in Xenograft Models

Generally, the ability of treatment with a first agent that inhibits the activity of HGF (i.e., an HGF antagonist or cMet antagonist), in combination with a second agent that is a PTEN agonist, to inhibit growth of human tumors is demonstrated in xenograft models in immunodeficient mice or other rodents such as rat. Illustrative but not limiting examples of immunodeficient strains of mice that can be used are nude mice such as CD-1 nude, Nu/Nu, Balb/c nude, NIH-III (NIH-bg-nu-xid BR); scid mice such as Fox Chase SCID (C.B-17 SCID), Fox Chase outbred SCID and SCID Beige; mice deficient in RAG enzyme; as well as nude rats. Experiments are carried out as described previously (Kim et al., Nature 362:841, 1992, which is incorporated herein by reference). Human tumor cells typically grown in complete DMEM medium are typically harvested in HBSS. Female immunodeficient, e.g., athymic nude mice (4-6 wks old) are injected s.c. with typically 5×10⁶ cells in 0.2 ml of HBSS in the dorsal areas. When the tumor size reaches 50-100 mm³, the mice are grouped randomly and appropriate amounts of the agents are administered. Alternatively, in the case of brain tumor cells such as gliomas (e.g., U87 cell line), the tumor cells may be implanted intracranially as has been described (see Kim et al., op. cit., 2006 and WO 06130773 A2), and treatment initiated on day 0, 7, 14 or other suitable time.

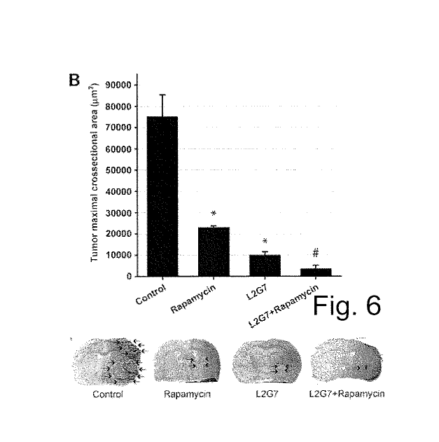

For example, wild-type U87 glioblastoma cells (3×10⁵) were implanted stereotactically in immunodeficient mice brains (n=10). One week post-tumor implantation, the animals were treated with i.p. injections of L2G7 (100 μg twice per week), the mTOR inhibitor rapamycin (40 μg three times per week), L2G7+rapamycin (same dose schedule as when used individually), or vehicle control for 2 weeks. The animals were euthanized 1 week after the last treatment and tumor cross-sectional areas were measured on H&E-stained brain cross-sections with computer-assisted image analysis (FIG. 6). Systemic L2G7 and rapamycin single treatments significantly inhibited tumor growth relative to control (P<0.05 relative to control). Combined systemic delivery of L2G7 and rapamycin led to significantly greater inhibition of xenograft growth than single treatments (P<0.05, relative to single treatment). Therefore, combination therapy of L2G7 and rapamycin led to an additive inhibitory effect on in vivo tumor growth over single therapies.

More generally, once treatment is initiated, an anti-HGF or other mAb (typically between 0.1 and 1.0 mg, e.g. 0.5 mg) is administered i.p. once, twice or three times per week in a volume of, e.g., 0.1 ml, for e.g., 1, 2, 3, or 4 weeks or the duration of the experiment. An orally active small molecule agent may be administered in drinking water or by injection. For s.c. tumors, tumor sizes are determined typically twice a week by measuring in two dimensions [length (a) and width (b)]. Tumor volume is calculated according to V=ab²/2 and expressed as mean tumor volume±SEM. The number of mice in each treatment group is at least 3, but more often between 5 and 10, e.g., 7. One group of mice is treated with both agents; other groups may be treated with neither agent or with one agent but not the other agent. Omitted agents may optionally be substituted by a “placebo” of like kind, e.g., an irrelevant mAb instead of an active mAb. Statistical analysis may be performed using, e.g., Student's t test. In a variation of this experiment, administration of the agents begins simultaneously or shortly after injection of the tumor cells. The effect of the agents may be measured by growth of the tumor with time, prolongation of the survival of the mice, or increase in percent of the mice surviving at a given time or indefinitely. Survival is an especially useful endpoint when the tumors were implanted intracranially.

Various tumor cell lines known to secrete or respond to HGF are used in separate experiments, for example U87 or U 118 human glioblastoma cells, and/or GB-dl human gallbladder tumor cells. Preferably, the cells are also deficient in PTEN. Preferred mAbs to be used as the first agent are neutralizing anti-HGF mAbs that are human-like and/or have reduced-immunogenicity, such as the L2G7 mAb and its chimeric and humanized forms and mAbs with the same epitope as L2G7. Preferred second agents are mTOR inhibitors such as sirolimus or temsirolimus. The combination of first and second agents inhibits the growth of tumor xenografts by at least 25%, but possibly 40% or 50%, and as much as 75% or 90% or greater, or even completely inhibits tumor growth after some period of time or causes tumor regression or disappearance. There may also be this extent of increased inhibition when both agents are used compared to only one. This inhibition takes place for at least tumor cell lines such as U87 or U118 in at least one mouse strain such as NIH III Beige/Nude, but preferably occurs for 2, 3, several, many, or even essentially all HGF-expressing tumor cell lines of a particular (e.g., glioma) or any type, when tested in one or more immunodeficient mouse strains that do not generate a neutralizing antibody response against the injected antibody. Treatment with some combinations of first and second agents in one or more of the xenograft models prolongs median survival by at least 25%, 50% or 100% and/or leads to the indefinite survival of 50%, 75%, 90% or even essentially all mice, who would otherwise die or need to be sacrificed.

Similar tumor inhibition experiments are performed with the HGF inhibitor (e.g., L2G7) and PTEN agonist (e.g., mTOR inhibitor) administered together with one or more chemotherapeutic agents (see supra) to which the tumor type is expected to be responsive, as described by Ashkenize et al., J. Clin. Invest. 104:155, 1999. The combination of the two agents and chemotherapeutic drug may produce a greater inhibition of tumor growth than either the agents or chemotherapy alone. The effect may be additive or synergistic, and strongly inhibit growth, e.g. by 80% or 90% or more, or even cause tumor regression or disappearance. The HGF inhibitor and PTEN agonist may also be administered in combination with an antibody against another growth or angiogenic factor, for example anti-VEGF or anti-EGFR, to obtain additive or synergistic growth inhibition and/or tumor regression or disappearance.

### 8. Antitumor Efficacy of a Humanized L2G7 (HuL2G7) in Combination with Rapamycin in Human Glioblastoma U-87 MG Tumor Xenograft Model

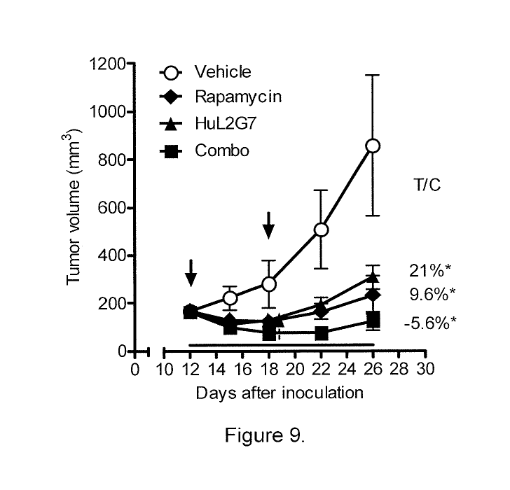

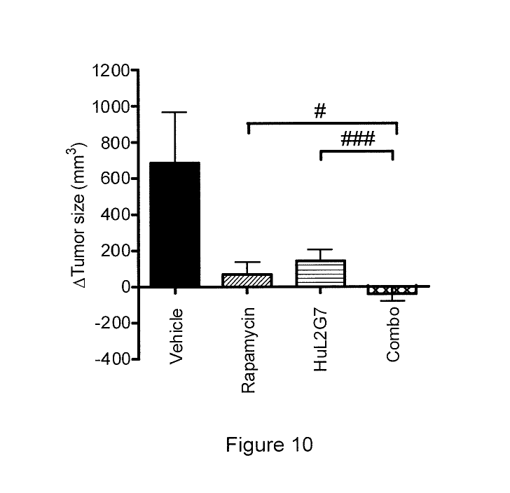

The combination effect of HuL2G7 with Rapamycin was examined in human glioblastoma U-87 MG tumor xenograft model. Female athymic nude mice (BALB/c-nu/nu, 30 animals) were purchased from Charles River Laboratories (Kanagawa, Japan). Mice were housed in a barrier facility with 12-h light/dark cycles and provided with food and tap water ad libitum and maintained for three weeks. U-87 MG cells (2.5×10⁶ in 100 μL) were suspended with 50% Matrigel (BD Biosciences) buffered with Hanks. balanced salt solution (Invitrogen Corp, Carlsbad, Calif., USA) and inoculated subcutaneously into the right-flank of each mouse. After the tumor xenografts were established, at day 12 after inoculation, mice with tumors (approximately 95-272 mm³) were randomized into groups of 4 (n=6) based on tumor volumes so as to minimize variation in tumor volumes. Then each group was treated with vehicle, HuL2G7, Rapamycin, or both drugs. HuL2G7 was administered intravenously with a dose of 0.5 mg/kg on days 12 and 18. Rapamycin was administered intraperitoneally and once-daily at a dose of 0.1 mg/kg on days 12-26. Administration volume was 10 mL/kg. HuL2G7 was stored in a refrigerator and diluted in saline at room temperature just before injection. There were no abnormalities in general observations of all animals. Tumor volumes were assessed by bilateral vernier caliper measurement at days 12, 15, 18, 22, and 26 after inoculation and calculated using the formula, length×width²×½, where length was taken to be the longest diameter across the tumor and width the corresponding perpendicular. Tumor growth was evaluated by the following formula: T/C (%)=ΔT/ΔC×100, where ΔT and ΔC are changes in tumor volume for each treated and vehicle control group. The T/C values on days 26 of mice treated with HuL2G7 alone, Rapamycin alone, and a combination of the two were 21, 9.6, and −5.6%, respectively (FIG. 9). Significant difference of ΔT values between the mice group treated with a combination of the two drugs and the mice group treated with HuL2G7 alone were observed by Student's t-test (P<0.05, FIG. 10). In addition, significant difference of ΔT values between the mice group treated with a combination of the two drugs and the mice group treated with Rapamycin alone were also observed (P<0.05). As a result, administration of HuL2G7 in combination with Rapamycin exerted stronger antitumor activity than each single treatment in U-87 MG glioblastoma mice xenograft model.

### 9. Sequences of Preferred Anti-HGF Mabs for Use in the Invention

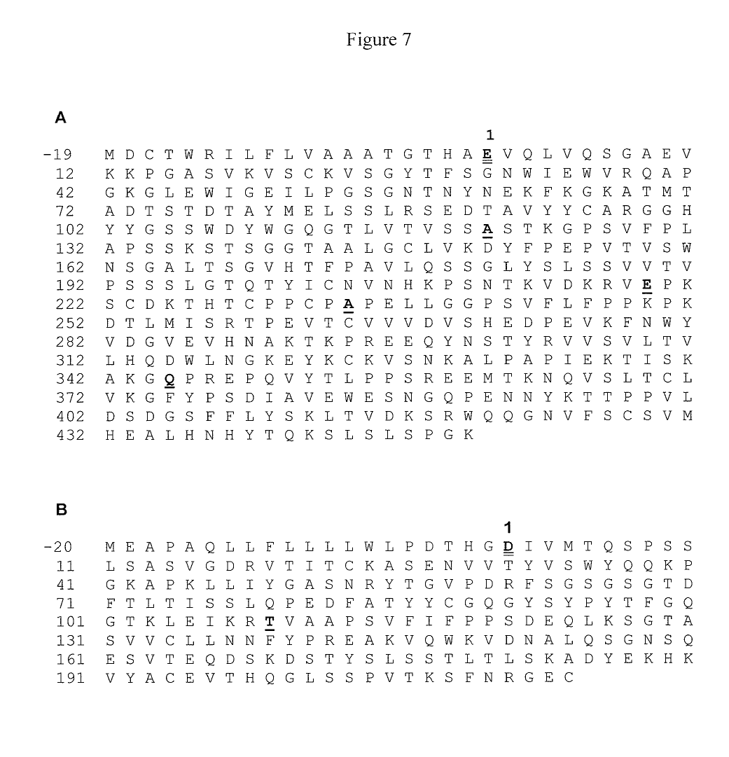

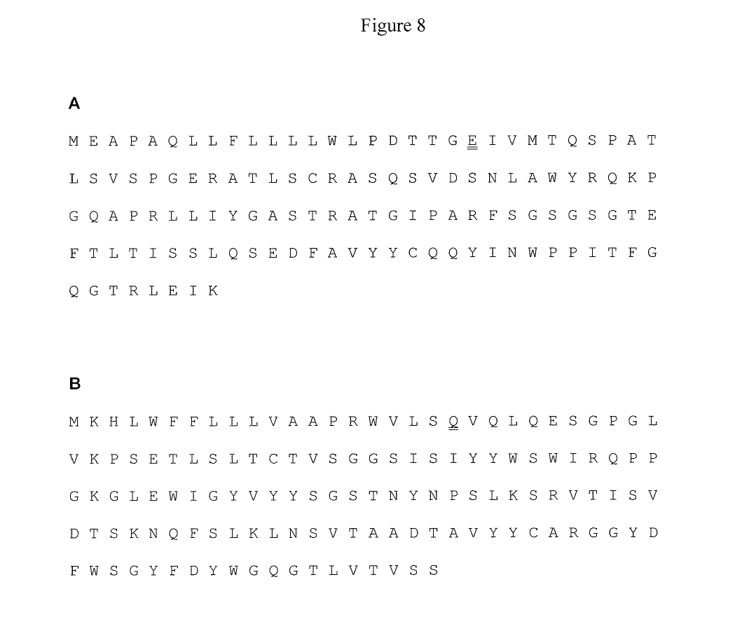

As mentioned above, a humanized form of the neutralizing anti-HGF mAb L2G7, e.g., HuL2G7, is especially preferred as the first agent in the invention. The sequences of the heavy and light chains of HuL2G7 are shown in FIG. 7, with the first amino acid of the mature sequences (i.e., the first amino acids of the actual mAb HuL2G7) double underlined. The C-terminal lysine in the heavy chain can be removed in expression and secretion and may therefore be absent in the final product. Also especially preferred for use as the first agent is the anti-HGF mAb 2.12.1 described in WO 2005/017107 A2; the sequences of the variable regions of the light and heavy chains of this mAb are shown in FIG. 8 with the first amino acid of the mature sequences (i.e., the first amino acids of the actual mAb 2.12.1) double underlined. The 2.12.1 mAb has as human constant regions adjoined to these light and heavy chain variable region sequences the human kappa constant region and the human gamma-2 constant region respectively, but mAbs with these variable regions and other human constant regions such as gamma-1 are also preferred for use in the invention. MAbs having light and heavy chain variable regions with the same CDRs as those shown in FIG. 7 or FIG. 8 are also preferred for use in the invention. MAbs that have amino acid sequences 90%, 95% or 99% identical to those shown in FIG. 7 or FIG. 8, at least in the CDRs, when aligned according to the Kabat numbering convention, or which differ from FIG. 7 or FIG. 8 by a small number of functionally inconsequential amino acid substitutions (e.g., conservative substitutions), deletions, or insertions, can also be used in the invention, provided they maintain the functional properties of HuL2G7 or 2.12.1 respectively.

Although the invention has been described with reference to the presently preferred embodiments, it should be understood that various modifications can be made without departing from the invention. Unless otherwise apparent from the context any step, element, embodiment, feature or aspect of the invention can be used with any other.

All publications (including GenBank Accession numbers, UniProtKB/Swiss-Prot accession numbers and the like), patents and patent applications cited are herein incorporated by reference in their entirety for all purposes to the same extent as if each individual publication, patent and patent application was specifically and individually indicated to be incorporated by reference in its entirety for all purposes. In the event of any variance in sequences associated with Genbank and UniProtKB/Swiss-Prot accession numbers and the like, the application refers to the sequences associated with the cited accession numbers as of the priority date of the application (Apr. 11, 2008).

U.S. Application Nos. 61/044,440 and 61/044,444 filed Apr. 11, 2008 and PCT application attorney dockets 022382-000510PC and 022382-000610PC filed on the same day as the present application, are also directed to methods of treating cancer by combination of inhibitors of HGF and a second agent inhibiting a second pathway. Unless otherwise apparent from the context, any step, element, embodiment, feature or aspect of the present application can be combined with any step, element, embodiment, feature or aspect of U.S. Ser. Nos. 61/044,440, 61/044,444, or attorney dockets 022382-000510PC and 022382-000610PC, all of which are incorporated by reference.

ATCC Number PTA-5162 has been deposited at the American Type Culture Collection, P.O. Box 1549 Manassas, Va. 20108, as ATCC Number PTA-5162 under the Budapest Treaty. This deposit will be maintained at an authorized depository and replaced in the event of mutation, nonviability or destruction for a period of at least five years after the most recent request for release of a sample was received by the depository, for a period of at least thirty years after the date of the deposit, or during the enforceable life of the related patent, whichever period is longest. All restrictions on the availability to the public of these cell lines will be irrevocably removed upon the issuance of a patent from the application.

We claim:

1. A method of treating cancer in a patient by administering to the patient a first agent that is an inhibitor of Hepatocyte Growth Factor (HGF) in combination with a second agent that is an agonist of PTEN.

2. The method of claim 1 wherein said first agent is a monoclonal antibody.

3. The method of claim 2 wherein the monoclonal antibody binds to and neutralizes HGF as a single agent.

4. The method of claim 3 wherein the monoclonal antibody is genetically engineered.

5. The method of claim 4 wherein the monoclonal antibody is human.

6. The method of claim 4 wherein the monoclonal antibody is humanized.

7. The method of claim 6 wherein the monoclonal antibody is a humanized L2G7 antibody.

8. The method of claim 1 wherein the second agent stimulates expression of PTEN.

9. The method of claim 1 wherein the second agent stimulates the activity of PTEN.

10. The method of claim 1 wherein the second agent is an mTOR inhibitor.

11. The method of claim 10 wherein the mTOR inhibitor is rapamycin.

12. The method of claim 1 wherein the cancer is glioma.

13. The method of claim 10 wherein the cancer is glioma.

14. A method of treating cancer in a patient by administering to the patient a first agent that is an inhibitor of Hepatocyte Growth Factor (HGF) in combination with a second agent that is an inhibitor of mTOR.

15. The method of claim 14 wherein said first agent is a monoclonal antibody that binds to and neutralizes HGF as a single agent.

16. The method of claim 15 wherein the monoclonal antibody is genetically engineered.

17. The method of claim 16 wherein the monoclonal antibody is human or humanized.

18. The method of claim 17 wherein the monoclonal antibody is a humanized L2G7 antibody.

19. The method of claim 14 wherein the inhibitor of mTOR is sirolimus or temsirolimus.

20. The method of claim 14 wherein the cancer is glioma.

21. A kit comprising a humanized L2G7 antibody and a PTEN agonist selected from the group consisting of rapamycin, sirolimus and temsirolimus.
